(19) United States
(12) Patent Application Publication
(10) Pub. No.: US 2019/0090732 A1
(43) Pub. Date: Mar. 28, 2019

(54) OPHTHALMIC APPARATUS, OPHTHALMIC IMAGE PROCESSING METHOD AND RECORDING MEDIUM

(71) Applicant: TOPCON CORPORATION, Tokyo (JP)

(72) Inventors: Wataru OYAGI, Tokyo (JP); Naoki INUZUKA, Tokyo (JP)

(73) Assignee: TOPCON CORPORATION, Tokyo (JP)

(21) Appl. No.: 16/140,873

(22) Filed: Sep. 25, 2018

(30) Foreign Application Priority Data
Sep. 27, 2017 (JP) 2017-186713

(51) Int. Cl. A61B 3/00; A61B 3/10; A61B 3/12

(52) U.S. Cl. CPC A61B 3/0025; A61B 3/102; A61B 3/0041; A61B 3/1241; A61B 3/1233

(57) ABSTRACT

An ophthalmic apparatus of an exemplary embodiment includes a storage and a difference processor. The storage stores a plurality of pieces of angiographic image data acquired by applying optical coherence tomography (OCT) angiography to the fundus of a subject's eye a plurality of times. The difference processor generates difference data between the first angiographic image data and the second angiographic image data both read out from the storage.

### CROSS-REFERENCE TO RELATED APPLICATION

This application is based upon and claims the benefit of priority from Japanese Patent Application No. 2017-186713, filed Sep. 27, 2017; the entire contents of which are incorporated herein by reference.

### FIELD

Embodiments described herein relate to an ophthalmic apparatus, an ophthalmic image processing method, and a recording medium.

### BACKGROUND

Diagnostic imaging serves an important role in the field of ophthalmology. In recent years, utilization of optical coherence tomography (OCT) has advanced. OCT is being used not only for acquiring B-scan images and three dimensional images of a subject's eye but also for acquiring front images (enface images) such as C-scan images and shadowgrams. OCT is also utilized for acquiring images in which a specific site of the subject's eye is emphasized and acquiring functional information.

For example, B-scan images and/or front images in which eye fundus blood vessels (retinal blood vessels, choroidal blood vessels) are emphasized can be constructed based on time series volume data (sequential volume data) acquired by OCT. This technique is disclosed, for example, in Japanese Unexamined Patent Application Publication (Translation of PCT Application) No. 2015-515894). This technique is referred to as OCT angiography (OCTA). B-scan images and/or front images in which eye fundus blood vessels are emphasized are referred to as blood vessel enhanced images, angiographic images, angiograms, motion contrast images, or the like.

### BRIEF DESCRIPTION OF THE DRAWINGS

FIG. 1 is a schematic diagram illustrating an example of the configuration of an ophthalmic apparatus according to an exemplary embodiment.

FIG. 2 is a flowchart illustrating an example of the operation of the ophthalmic apparatus according to the exemplary embodiment.

FIG. 3 is a schematic diagram illustrating an example of the configuration of the ophthalmic apparatus according to an exemplary embodiment.

FIG. 4A is a schematic diagram for describing an example of the operation of the ophthalmic apparatus according to the exemplary embodiment.

FIG. 4B is a schematic diagram for describing an example of the operation of the ophthalmic apparatus according to the exemplary embodiment.

FIG. 5 is a schematic diagram illustrating an example of the configuration of the ophthalmic apparatus according to an exemplary embodiment.

### DETAILED DESCRIPTION

The first aspect of exemplary embodiments is an ophthalmic apparatus that includes a storage and a difference processor. The storage stores a plurality of pieces of angiographic image data acquired by applying optical coherence tomography (OCT) angiography to a fundus of a subject's eye a plurality of times. The difference processor generates difference data between a first angiographic image data and a second angiographic image data both read out from the storage.

The second aspect of exemplary embodiments is the ophthalmic apparatus of the first aspect, further including a registration processor that performs registration between the first angiographic image data and the second angiographic image data, wherein the difference processor generates the difference data from the first angiographic image data and the second angiographic image data to which the registration has been applied.

The third aspect of exemplary embodiments is the ophthalmic apparatus of the first or the second aspect, further including a first information generation processor that generates blood flow change information representing a change in blood flow in the fundus based on the difference data generated by the difference processor.

The fourth aspect of exemplary embodiments is the ophthalmic apparatus of any of the first to the third aspects, further including a second information generation processor that generates blood vessel diameter change information representing a change in a blood vessel diameter in the fundus based on the difference data generated by the difference processor.

The fifth aspect of exemplary embodiments is the ophthalmic apparatus of any of the first to the fourth aspects, further including: a data acquisition device that acquires data by applying OCT angiography to the fundus; and a data processor that processes the data acquired by the data acquisition device to construct angiographic image data, wherein the storage stores the angiographic image data constructed by the data processor.

The sixth aspect of exemplary embodiments is the ophthalmic apparatus of any of the first to the fifth aspects, further including: an observation system that provides an observation image of the fundus to a user; an irradiation system that irradiates the fundus with treatment laser light; and a display controller that displays an irradiation target position image indicating an irradiation target position of the treatment laser light and a difference image constructed from the difference data generated by the difference processor on a display device, wherein the irradiation target position image and the difference image are provided together with the observation image to the user.

The seventh aspect of exemplary embodiments is a method of processing an ophthalmic image, the method including: storing a plurality of pieces of angiographic image data acquired by applying optical coherence tomography (OCT) angiography to a fundus of a subject's eye a plurality of times; and generating difference data between a first angiographic image data and a second angiographic image data from among the plurality of pieces of angiographic image data.

A non-transitory computer readable recording medium storing a program causing a computer to execute the ophthalmic image processing method.

Exemplary embodiments will be described with referring to the drawings. It should be noted that any of the matters and items disclosed in the documents cited in the present specification as well as any known techniques and technologies can be incorporated into the embodiments.

Ophthalmic apparatuses, ophthalmic image processing methods, programs, and recording mediums according to the exemplary embodiments will be described below. An exemplary ophthalmic image processing method can be realized with an exemplary ophthalmic apparatus. Further, an exemplary ophthalmic image processing method can be executed according to an exemplary program.

The exemplary ophthalmic apparatus may be a single device (e.g., a computer including a storage device), or may include two or more devices (e.g., one or more computers, one or more storage devices) that can communicate with each other.

The exemplary ophthalmic apparatus may have at least one of a function of imaging the subject's eye, a function of measuring a characteristic of the subject's eye, and a function of treating the subject's eye. OCT apparatuses, fundus cameras, scanning laser ophthalmoscopes, slit lamp microscopes, and surgical microscopes are examples of ophthalmic apparatuses having the imaging function. Refractometers, keratometers, tonometers, wave front analyzers, specular microscopes, and perimeters are examples of ophthalmic apparatuses having the measuring function. Laser treatment apparatuses and cataract surgery apparatuses are examples of ophthalmic apparatuses used for medical treatment.

Hardware and software for realizing the exemplary ophthalmic image processing method is not limited to the ophthalmic apparatus described below as an example, but may include a combination of any hardware and any software which contributes to realization of the exemplary ophthalmic image processing method. The software includes the exemplary program.

The exemplary program causes a computer included in the exemplary ophthalmic apparatus (or another computer) to perform the exemplary ophthalmic image processing method. The exemplary recording medium is a computer-readable recording medium, and records the exemplary program. The exemplary recording medium is a non-transitory recording medium. The exemplary recording medium may be an electronic medium utilizing magnetic technique, optical technique, magneto-optical technique, semiconductor technique, or other techniques. Typically, the exemplary recording medium is a magnetic tape, a magnetic disk, an optical disk, a magneto-optical disk, a flash memory, a solid state drive, or another type of recording medium.

### First Embodiment

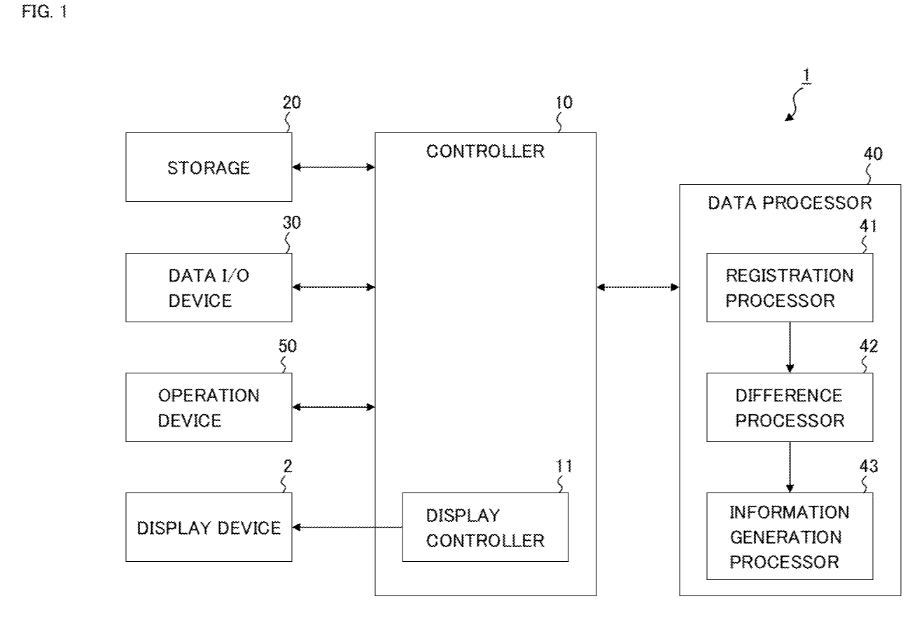

The ophthalmic apparatus according to the first embodiment will be described. The ophthalmic apparatus 1 shown in FIG. 1 can display various kinds of information including images of the fundus of the subject's eye on the display device 2. The display device 2 may be a part of the ophthalmic apparatus 1 or may be an external device connected to the ophthalmic apparatus 1.

The ophthalmic apparatus 1 includes the controller 10, the storage 20, the data input and output device (data I/O device) 30, the data processor 40, and the operation device 50.

### (Controller 10)

The controller 10 controls each part of the ophthalmic apparatus 1. The controller 10 includes a processor. In this specification, the term “processor” is used to mean, for example, a circuit such as a central processing unit (CPU), a graphics processing unit (GPU), an application specific integrated circuit (ASIC), a programmable logic device (for example, a simple programmable logic device (SPLD), a complex programmable logic device (CPLD), or a field programmable gate array (FPGA)), or the like. The controller 10 realizes the functions according to the embodiment, for example, by reading out and executing a program(s) stored in a storage circuit or a storage device (for example, the storage 20).

The controller 10 includes the display controller 11. The display controller 11 executes processing for causing the display device 2 to display information.

### (Storage 20)

The storage 20 stores various kinds of information. In the present example, the storage 20 stores a plurality of pieces of angiographic image data acquired by applying an OCT angiography to the fundus of the subject's eye a plurality of times. The storage 20 includes a storage device such as a hard disk drive.

In a typical example, the plurality of pieces of angiographic image data are acquired in follow-up observation, preoperative postoperative comparison, checking of the effect of administered medicine, health examination, health checkup, or other types of medical practice or activity. For example, the plurality of pieces of angiographic image data may include a data group acquired on a plurality of different days in follow-up observation. The plurality of pieces of angiographic image data may include both data acquired before surgery or laser treatment and data acquired after the surgery or the laser treatment.

Although not illustrated in figures, the storage 20 of a typical embodiment stores templates of screens, dialogs, icons, and other types of objects, which are displayed as GUI on the display device 2. The storage 20 stores a program executed for data processing and a program executed for controlling the GUI. The processing according to the present embodiment is realized by the cooperation of software including such programs and hardware including one or more processors.

### (Data Input and Output Device 30)

The data input and output device 30 performs input of data into the ophthalmic apparatus 1 and output of data from the ophthalmic apparatus 1. The data input and output device 30 may include a communication device for transmitting and receiving data via a communication line such as a local area network (LAN), the Internet, a dedicated line, etc. The data input and output device 30 may include a reader writer device for reading data from a recording medium and writing data into a recording medium. Further, the data input and output device 30 may include an image scanner that scans information recorded on a print medium or the like, a printer that records information on a paper medium, or other types of device.

### (Data Processor 40)

The data processor 40 includes a processor and executes various kinds of data processing. For example, the data processor 40 applies image processing to ophthalmic image data. The data processor 40 includes the registration processor 41, the difference processor 42, and the information generation processor 43.

### (Registration Processor 41)

The registration processor 41 performs registration between the first angiographic image data and the second angiographic image data from among the plurality of pieces of angiographic image data stored in the storage 20. In the present example, angiographic image data is typically three dimensional angiographic image data or rendered image data constructed from the three dimensional angiographic image data.

Registration is alignment or position matching between two or more different images (image matching). Any registration method can be applied to the present embodiment. For example, a feature-based registration method or an area-based registration method is applied. The feature-based method extracts feature points (e.g., edges, corners) from images, calculates feature amounts from information surrounding the feature points, and performs registration between the images based on the feature amounts. The area-based method prepares a template of an area to be searched, and performs registration between the images by comparing the template and the images.

When registration is performed between two dimensional image data (e.g., rendered image data) and three dimensional data (e.g., three dimensional angiographic image data), the present embodiment may execute preprocessing on one or both pieces of image data. For example, two dimensional angiographic image data (e.g., projection image data, shadowgram data, C-scan image data) can be generated by rendering three dimensional angiographic image data, and registration can be performed between the rendered two dimensional angiographic image data and two dimensional image data.

The difference processing described below may be executed without registration in the cases as the followings: when registration between the first angiographic image data and the second angiographic image data has already been performed by another apparatus; when the fixation of the subject's eye has been properly performed both at the time of acquisition of the first angiographic image data and at the time of acquisition of the second angiographic image data; when one of the first angiographic image data and the second angiographic image data has been acquired using the other as a standard; or when the imaging area of the first angiographic image data and the imaging area of the second angiographic image data have been matched using image processing such as trimming.

### (Difference Processor 42)

The difference processor 42 generates difference data between the first angiographic image data and the second angiographic image data.

The difference data generating process includes, for example, processing of calculating the difference between the value (brightness value) of each pixel of the first angiographic image data and the value (brightness value) of the corresponding pixel of the second angiographic image data.

The sign (positive or negative, plus or minus) of the difference between brightness values (brightness difference) may be taken into account in the brightness difference calculating process. Alternatively, the brightness difference calculating process may include the process of determining the absolute value of a brightness difference.

When taking the sign of the brightness difference into account, all the pixels of the difference data (image data) are classified into pixels with positive difference, pixels with negative difference, and pixels with zero difference. When the first angiographic image data is acquired before the second angiographic image data and the first angiographic image data is subtracted from the second angiographic image data, the following correspondences can be made in principle: pixels with positive difference correspond to positions where blood flow has increased over time; pixels with negative difference correspond to positions where blood flow has decreased over time; and pixels with zero difference correspond to positions where blood flow has been unchanged over time, or to non-blood vessel positions.

In the case of determining the absolute value of the brightness difference, all the pixels of the difference data are classified into pixels with non-zero absolute difference (positive value) and pixels with zero absolute difference. When the first angiographic image data is acquired before the second angiographic image data and the first angiographic image data is subtracted from the second angiographic image data, the following correspondences can be made in principle: pixels with non-zero difference correspond to positions where blood flow has changed over time; and pixels with zero difference correspond to positions where blood flow has been unchanged over time, or to non-blood vessel positions.

### (Information Generation Processor 43)

The information generation processor 43 generates medical information based on the difference data generated by the difference processor 42. Typical medical information generated by the information generation processor 43 is information that can be referred to for diagnosis of the subject's eye.

In one example, the information generation processor 43 can generate information indicating changes in blood flow (blood flow change information) in the fundus of the subject's eye, based on the difference data generated by the difference processor 42. A typical example of the blood flow change information is a map in which pixel values of the difference data are expressed in color. The map is hereinafter referred to as a blood flow change map.

The blood flow change map may be created by assigning different colors depending on the signs (positive or negative) of the pixel values of the difference data. The blood flow change map may be created by expressing the changes in the magnitudes of the pixel values of the difference data in a stepwise or continuous manner.

Such a blood flow change map can express the temporal change in a blood flow parameter such as blood flow amount or blood flow velocity. For example, a blood flow change map can express an event that blood flow has stopped, an event that blood flow has started, an event that blood flow has decreased, an event that blood flow has increased, or other events.

The blood flow change map can also be generated by determining a blood flow parameter map from the first angiographic image data and another blood flow parameter map from the second angiographic image data, and by calculating the difference between blood flow parameters at corresponding pixel positions of the blood flow parameter maps.

In another example, the information generation processor 43 can generate information indicating changes in blood vessel diameters (blood vessel diameter change information) in the fundus of the subject's eye, based on the difference data generated by the difference processor 42.

The calculation of a blood vessel diameter includes, for example, a process of determining a blood vessel axis line by applying thinning to a blood vessel image, and a process of calculating the width of the blood vessel (blood vessel diameter) in a direction perpendicular to the blood vessel axis line based on the number of pixels.

From the principle of OCT angiography, the blood vessel diameter change information represents the change in the inner diameter of a blood vessel. A typical example of the blood vessel diameter change information is a map in which the magnitudes of the changes in blood vessel diameters are expressed in color. The map is hereinafter referred to as a blood vessel diameter change map.

The blood vessel diameter change map may be created by assigning different colors respectively to areas where blood vessel diameter has increased, areas where blood vessel diameter has decreased, and areas where blood vessel diameter is unchanged. The blood vessel diameter change map may be created by expressing the changes in the magnitudes of blood vessel diameters in a stepwise or continuous fashion.

The blood vessel diameter change map can also be generated by determining blood vessel diameters from the first angiographic image data and blood vessel diameters from the second angiographic image data, and by calculating the difference between blood vessel diameters at corresponding pixel positions of the first and second angiographic image data.

Information that can be generated by the information generation processor 43 is not limited to the above examples, and may be any information that can be derived by processing difference data, or any information that can be derived by processing two or more pieces of angiographic image data.

Another process that can be executed by the data processor 40 will be described. The data processor 40 is capable of performing rendering such as three dimensional computer graphics (3DCG).

For example, when a three dimensional data set (e.g., volume data, stack data) of the subject's eye has been input to the ophthalmic apparatus 1, the data processor 40 can apply various kinds of rendering to the three dimensional data set to generate a B-scan image (longitudinal cross sectional image, axial cross sectional image), a C-scan image (transverse cross sectional image, horizontal cross sectional image), a projection image, a shadowgram, and other images. An image of any cross section such as a B-scan image or a C-scan image is constructed by, for example, selecting image elements (pixels, voxels) on a designated cross section from the three dimensional data set. An image of any cross section can be constructed from a slice of the three dimensional data set. A projection image is constructed by projecting the three dimensional data set in a predetermined direction (Z direction, depth direction, axial direction). A shadowgram is constructed by projecting part of the three dimensional data set (e.g., partial data corresponding to a specific layer) in a predetermined direction. An image that is viewed from the front side of the subject's eye, such as a C-scan image, a projection image, or a shadowgram, is referred to as a front image or an enface image.

The data processor 40 is capable of executing various types of image processing in addition to rendering. For example, the data processor 40 may be capable of performing segmentation to determine a specific tissue or a specific tissue boundary, and size analysis to determine the size of a specific tissue (e.g., layer thickness, volume). When a specific layer (or a specific layer boundary) is determined by segmentation, a B-scan image or a front image can be reconstructed so that the specific layer becomes flat. Such an image is called a flattened image.

### (Operation Device 50)

The operation device 50 is used by the user to input instructions and information to the ophthalmic apparatus 1. The operation device 50 may include a known operation device usable together with a computer. For example, the operation device 50 may include a pointing device such as a mouse, a touch pad, or a track ball. The operation device 50 may include a keyboard, a pen tablet, a dedicated operation panel, or other devices.

### (Angiographic Image Data)

As described above, the ophthalmic apparatus 1 processes angiographic image data. Angiographic image data is an image constructed by the following processes: a process of analyzing an image acquired by OCT scan to specify image regions (blood vessel regions) corresponding to blood vessels; and a step of changing the expression modes (expression aspects) of the blood vessel regions to emphasize the blood vessel regions. The specification of the blood vessel regions uses a plurality of images acquired by repeatedly applying OCT scans to substantially the same area of the subject's eye. In the present embodiment, for example, the storage 20 stores three dimensional angiographic image data, angiographic image data as a front image, angiographic image data as a two dimensional cross sectional image, and other images. Alternatively, the storage 20 may store one or more three dimensional data sets for constructing angiographic image data. If this is the case, the ophthalmic apparatus 1 (e.g., the data processor 40) may include known hardware and known software for constructing angiographic image data from a three dimensional data set.

There are several types of angiographic image data constructing methods. A typical example thereof will be described. Note that at least part of the plurality of steps included in the method described below may be performed by the data processor 40. At least part of the plurality of steps included in the method described below may be performed by another apparatus (a computer, an ophthalmic apparatus, etc.).

First, by repeatedly scanning each of a plurality of B-scan cross sections of the fundus of the subject's eye, constructed is a three dimensional data set including a plurality of B-scan images arranged in time series (along the time axis) for each B-scan cross section. Fixation and tracking are known as methods for repeatedly scanning substantially the same B-scan cross section. The three dimensional data set at this stage may be stored in the storage 20.

Next, position matching of the plurality of B-scan images is performed for each B-scan cross section. The position matching is performed, for example, by using a known image matching technique. A typical example thereof may execute extraction of a feature region from each B-scan image, and position matching of the plurality of extracted feature regions to aligning the plurality of B-scan images. The three dimensional data set at this stage can be stored in the storage 20.

Subsequently, a process of specifying image regions that change between the plurality of B-scan images aligned is performed. This processing includes, for example, a process of determining the difference between different B-scan images. Each B-scan image is brightness image data representing the morphology (structure) of the subject's eye, and it can be considered that an image region therein corresponding to a site other than blood vessels is substantially invariant. On the other hand, considering that the backscattering contributing to interference signals randomly varies under the influence of blood flow, an image region in which a change has occurred between the plurality of B-scan images aligned can be regarded as a blood vessel region. Here, the image region with a change includes, for example, pixels with non-zero difference or pixels with difference equal to or larger than a predetermined threshold. The three dimensional data set at this stage can be stored in the storage 20.

To the image region specified in this way, information indicating that the image region is a blood vessel region can be assigned. In other words, the pixel values of the specified image region can be changed (processed). With this, angiographic image data is acquired. The angiographic image data can be stored in the storage 20.

### [Operation]

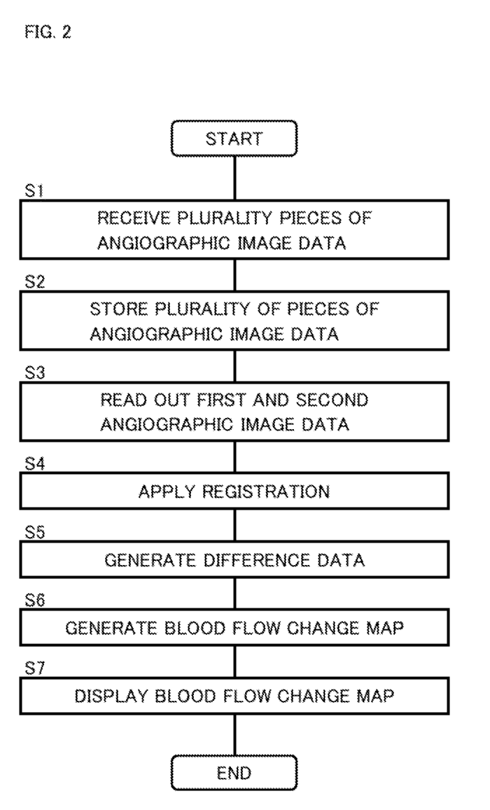

The operation of the ophthalmic apparatus 1 according to the exemplary embodiment will be described. FIG. 2 shows an exemplary flow of the operation of the ophthalmic apparatus 1.

### (S1: Receive Plurality of Pieces of Angiographic Image Data)

The ophthalmic apparatus 1 receives, by the data input and output device 30, a plurality of pieces of angiographic image data of the fundus of the subject's eye acquired on different days in follow-up observation or the like.

An OCT scan of the fundus in OCT angiography is performed using an ophthalmic imaging apparatus having the OCT function. The ophthalmic imaging apparatus or another apparatus performs construction of the plurality of pieces of angiographic image data based on data acquired by the OCT scan.

The plurality of pieces of angiographic image data constructed are sent directly or indirectly to the ophthalmic apparatus 1 or are stored in an image archiving apparatus, for example. In the latter case, the plurality of pieces of angiographic image data are then sent directly or indirectly from the image archiving apparatus to the ophthalmic apparatus 1. The ophthalmic apparatus 1, by the data input and output device 30, receives the plurality of pieces of angiographic image data transmitted from the ophthalmic imaging apparatus, the image archiving apparatus, or another apparatus.

### (S2: Store Plurality of Pieces of Angiographic Image Data)

The controller 10 stores the plurality of pieces of angiographic image data received in step S1 in the storage 20.

### (S3: Read Out First and Second Angiographic Image Data)

The controller 10 reads out the first angiographic image data and the second angiographic image data from among the plurality of pieces of angiographic image data from the storage 20 and sends them to the data processor 40.

The selection of the first angiographic image data and the second angiographic image data is performed by the user or the ophthalmic apparatus 1, for example.

When the user makes the selection, for example, the display controller 11 displays a list of the plurality of pieces of angiographic image data or thumbnails of the plurality of pieces of angiographic image data on the display device 2. Alternatively, the display controller 11 displays a plurality of angiographic images respectively generated from the plurality of pieces of angiographic image data side by side or in a switching manner. The user can refer to the displayed information and select desired angiographic image data using the operation device 50.

When the ophthalmic apparatus 1 makes the selection, for example, the controller 10 can select the first angiographic image data and the second angiographic image data based on information attached to the plurality of pieces of angiographic image data. Typically, the controller 10 can select the first angiographic image data and the second angiographic image data based on imaging date information attached to the plurality of pieces of angiographic image data. The imaging date information indicates the dates on which the plurality of pieces of angiographic image data has been acquired. As an example, the controller 10 can select the first angiographic image data and the second angiographic image data whose imaging dates are consecutive, or select the angiographic image data corresponding to the reference imaging date such as the first imaging date (that is, the angiographic image data as a baseline) and the latest angiographic image data.

The information attached to angiographic image data may be recorded in the DICOM tag of the angiographic image data, for example. DICOM is an abbreviation for “Digital Imaging and COmmunications in Medicine” which is the standard that defines medical image formats and communication protocols. DICOM tags are tag information provided in DICOM files.

### (S4: Apply Registration)

The registration processor 41 performs registration between the first angiographic image data and the second angiographic image data read out in step S3.

### (S5: Generate Difference Data)

The difference processor 42 generates difference data from the first angiographic image data and the second angiographic image data to which the registration has been applied in step S4.

### (S6: Generate Blood Flow Change Map)

The information generation processor 43 generates medical information based on the difference data generated in step S5. In the present example, a blood flow change map is generated.

### (S7: Display Blood Flow Change Map)

The display controller 11 displays the blood flow change map generated in step S6 on the display device 2.

### Second Embodiment

The ophthalmic apparatus according to the present embodiment has a configuration for applying optical coherence tomography (OCT) to the fundus of the subject's eye. In particular, the ophthalmic apparatus according to the present embodiment is capable of executing control and data processing for implementing OCT angiography. The OCT method may be, for example, spectral domain OCT or swept source OCT.

The spectral domain OCT is a method of constructing OCT image data by acquiring a spectrum of interference light in a space division manner using a broadband low coherence light source and a spectroscope, and by applying Fourier transform to the spectrum of the interference light acquired.

The swept source OCT is a method of constructing OCT image data by acquiring a spectrum of interference light in a time division manner using a wavelength swept light source (wavelength tunable light source) and a photodetector (e.g., a balanced photodiode), and by applying Fourier transform to the spectrum of the interference light acquired.

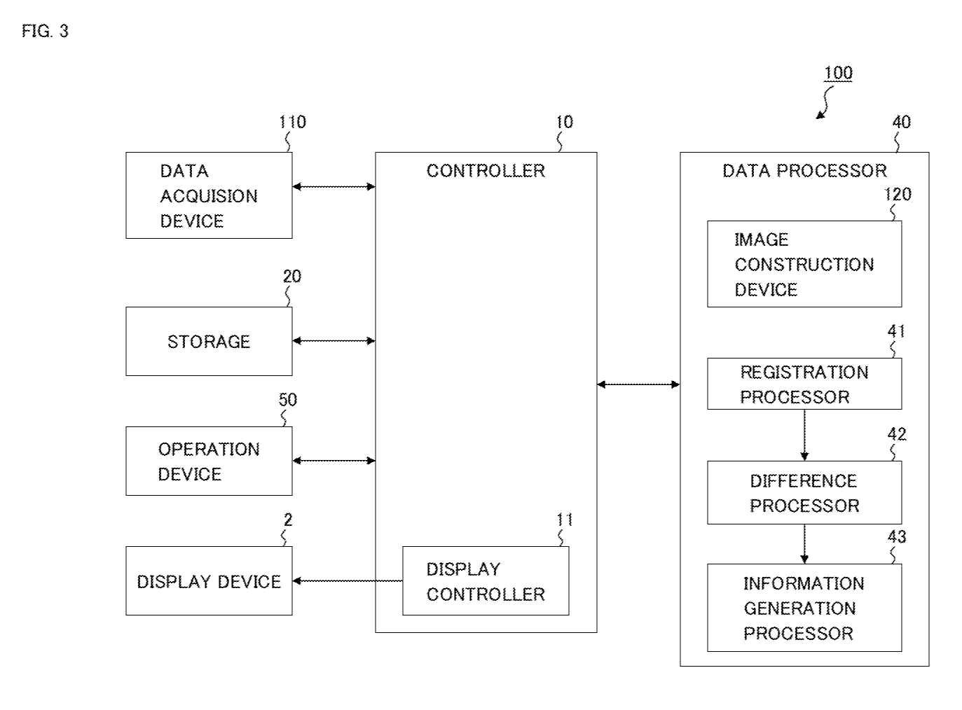

FIG. 3 shows an example of the configuration of the ophthalmic apparatus according to the present embodiment. Unlike the configuration of the first embodiment shown in FIG. 1, the ophthalmic apparatus 100 shown in FIG. 3 is provided with the data acquisition device 110 and the image construction device 120. The image construction device 120 is provided in the data processor 40.

The data acquisition device 110 applies OCT angiography to the eye fundus to acquire data. The image construction device 120 processes the data acquired by the data acquisition device 110 to construct angiographic image data. It should be noted that the data acquisition device 110 is capable of performing an ordinary OCT scan and the image construction device 120 is capable of constructing ordinary OCT image data.

The OCT angiography is typically a technique for constructing motion contrast image data (three dimensional angiographic image data) based on time series data acquired by applying OCT to a three dimensional region of the eye fundus.

The image construction device 120 includes an image constructing processor (not shown). The image construction device 120 constructs cross sectional image data of the eye fundus based on the data acquired by the data acquisition device 110. The image data construction includes signal processing such as noise removal (noise reduction), filtering, fast Fourier transform (FFT), and other processing, like a conventional OCT technique. Image data constructed by the image construction device 120 is a data set including a group of image data (a group of A-scan image data) constructed by applying image representation to the reflection intensity profiles of a plurality of A-lines (scan lines along the depth direction) arranged along a scan line.

When the OCT angiography is performed, the image construction device 120 can construct a motion contrast image based on data acquired by scans repeatedly performed a predetermined number of times. The motion contrast image is an angiographic image (angiogram) in which blood vessels of the eye fundus are emphasized. Note that a motion contrast image is an image that is generated based on a plurality of data (images) acquired at the same position at different times and that expresses movement at the position.

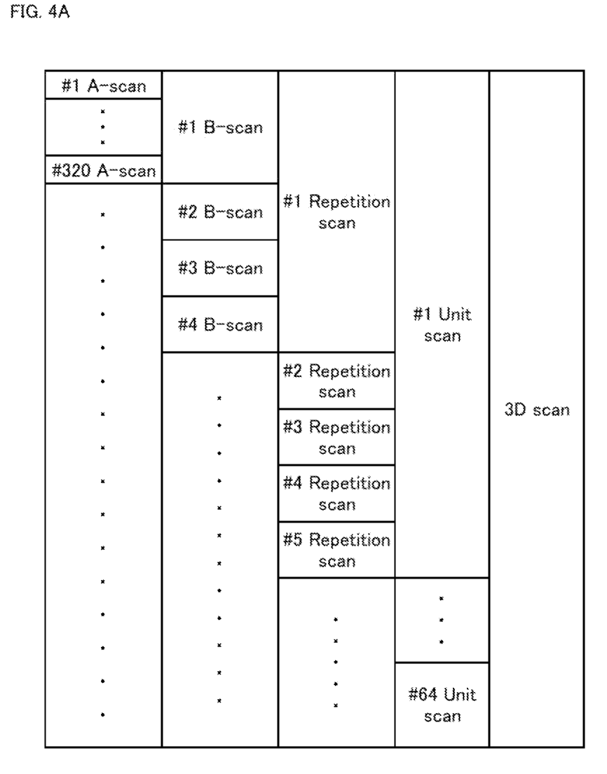

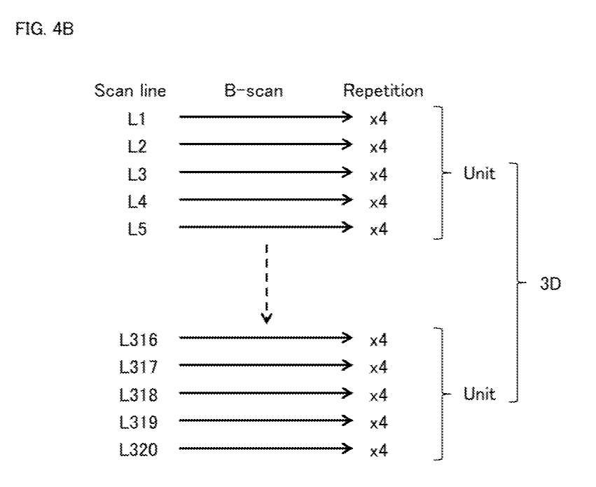

Here, a typical example of a scan pattern applicable to the OCT angiography will be described. The OCT angiography of the present example uses a three dimensional scan (raster scan). The three dimensional scan is a scan along a plurality of mutually parallel scan lines. The plurality of scan lines are ordered in advance, and the scan is applied in this order. An example of the three dimensional scan applicable in the present embodiment is shown in FIG. 4A and FIG. 4B.

As shown in FIG. 4B, the three dimensional scan of the present example is performed for 320 scan lines L1 to L320. One scan along one scan line Li (i=1 to 320) is called a B-scan. A single B-scan consists of 320 A-scans (see FIG. 4A). A single A-scan is a scan for one A-line. That is, a single A-scan is a scan for an A-line along the incident direction of the OCT measurement light (i.e., along the depth direction or the axial direction). A single B-scan consists of 320 A-line scans arranged along a scan line Li on the plane orthogonal to the depth direction.

In the three dimensional scan of the present example, B-scans are performed 4 times for each of the scan lines L1 to L320 according to a preset order. Four B-scans for each scan line Li is called a repetition scan. The order of 4 repetitions for each scan line Li is optional. For example, the 4 scans may be performed consecutively, or a B-scan for another scan line may be performed during the 4 scans.

The scan lines L1 to L320 are classified into a plurality of sets each consisting of 5 lines, according to the arrangement order of the scan lines L1 to L320. Each of the 64 sets obtained by the classification is called a unit, and scans for each unit are collectively called a unit scan. A unit scan consists of 4 B-scans (repetitions) for each of 5 scan lines. That is, a unit scan consists of 20 B-scans.

The image construction device 120 classifies the data acquired by the data acquisition device 110 in such a scan pattern, into data sets (time series data) for respective scan lines Li. Here, a data set includes 4 pieces of B-scan data corresponding to 4 repetitions. Each of the 4 pieces of B-scan data is data acquired by one B-scan for the scan line Li.

Further, the image construction device 120 constructs motion contrast image data corresponding to the scan line Li, based on the data set corresponding to the scan line Li. The motion contrast image data corresponding to the scan line Li is two dimensional angiographic image data representing a B-scan plane (longitudinal cross section) including the scan line Li.

The motion contrast image data construction is executed in the same manner as the conventional OCT angiography data construction. As described above, in the present example, 4 pieces of B-scan data are included in the data set corresponding to the scan line Li. Each B-scan data is data acquired by one B-scan for the scan line Li.

First, the image construction device 120 constructs ordinary OCT image data based on each B-scan data. The OCT image data is B-scan image data consists of 320 pieces of A-scan image data. As a result, 4 pieces of B-scan image data corresponding to the scan line Li are obtained.

Next, the image construction device 120 specifies image regions that change between the 4 pieces of B-scan image data. This processing includes, for example, a process of determining the difference between different pieces of B-scan image data. Each B-scan image data is brightness image data (intensity image data) representing the morphology of the eye fundus, and it can be considered that image regions therein corresponding to a site other than blood vessels is substantially invariant. On the other hand, considering that the backscattering contributing to interference signals randomly varies under the influence of blood flow, an image region in which a change has occurred between the 4 B-scan image data can be regarded as a blood vessel region. Here, the image region with a change includes, for example, pixels with non-zero difference or pixels with difference equal to or larger than a predetermined threshold.

The image construction device 120 assigns predetermined pixel values to the pixels in the blood vessel region specified. The pixel values may be, for example, relatively high brightness values (the pixels are expressed bright and in white when displayed) or a pseudo color values. Blood vessel regions can also be specified using Doppler OCT or image processing as in other conventional techniques.

Through such processing, obtained are 320 pieces of two dimensional angiographic image data corresponding to 320 scan lines L1 to L320. The image construction device 120 arranges the 320 pieces of two dimensional angiographic image data according to the arrangement of the 320 scan lines Li to L320. This processing includes, for example, a process of arranging (embedding) the 320 pieces of two dimensional angiographic image data in a single three dimensional coordinate system in accordance with the arrangement order and arrangement intervals (spacing) of the 320 scan lines L1 to L320. From this, stack data of the 320 pieces of two dimensional angiographic image data according to the arrangement of the 320 scan lines L1 to L320 can be constructed. The stack data is an example of image data representing a three dimensional distribution of blood vessels of the eye fundus. That is, stack data is an example of three dimensional angiographic image data. The image construction device 120 can also construct volume data (voxel data) by performing processing such as interpolation on the stack data.

The processing of constructing angiographic image data from the acquired data is not limited to the above example and angiographic image data can be constructed using any known technique.

The image construction device 120 can process three dimensional image data such as volume data or stack data. For example, the image construction device 120 may apply rendering to three-dimensional image data. Examples of the rendering method include volume rendering, maximum intensity projection (MIP), minimum intensity projection (MinP), surface rendering, and multi planar reconstruction (MPR). Further, the image construction device 120 can construct projection data or shadowgram data by projecting at least part of three dimensional image data in the direction along the A-lines (A-scan direction, depth direction).

The image construction device 120 can perform predetermined analysis processing and predetermined image processing. For example, the image construction device 120 can apply segmentation to two dimensional cross sectional image data or three dimensional image data. Segmentation is processing of specifying partial data in image data. The present example is capable of specifying an image region corresponding to a predetermined tissue of the eye fundus.

In OCT angiography, the image construction device 120 is capable of constructing any two dimensional angiographic image data and/or any pseudo three dimensional angiographic image data, from three dimensional angiographic image data. For example, the image construction device 120 can construct two dimensional angiographic image data representing a desired cross section of the eye fundus, by applying multi planar reconstruction to three dimensional angiographic image data.

Further, the image construction device 120 can specify an image region corresponding to a predetermined tissue of the eye fundus by applying segmentation to three dimensional angiographic image data, and project the specified image region in the A-scan direction to construct shadowgram data (front angiographic image data). Examples of the front angiographic image data include the followings: a front image data corresponding to any depth region of the eye fundus (e.g., a shallow portion of the retina, a deep portion of the retina, the choriocapillaris, the sclera); and front image data corresponding to a predetermined tissue of the eye fundus (e.g., inner limiting membrane, nerve fiber layer, ganglion cell layer, inner plexiform layer, inner nuclear layer, outer plexiform layer, outer nuclear layer, external limiting membrane, retinal pigment epithelium, Bruch's membrane, choroid, choroid-sclera interface, sclera, a part of any of these, a combination of at least two or more of these).

The storage 20 can store angiographic image data constructed by the image construction device 120. Further, the storage 20 can store image data constructed by rendering the angiographic image data constructed by the image construction device 120. In addition, as in the first embodiment, the storage 20 may store angiographic image data received from an external device. In this manner, the plurality of pieces of angiographic image data acquired by applying OCT angiography to the fundus of the subject's eye a plurality of times is stored in the storage 20.

The controller 10 reads out the first angiographic image data and the second angiographic image data from among the plurality of pieces of angiographic image data from the storage 20, and sends the data read out to the data processor 40. The registration processor 41 performs registration between the first angiographic image data and the second angiographic image data read out from the storage 20. The difference processor 42 generates difference data from the first angiographic image data and the second angiographic image data to which the registration has been applied. The information generation processor 43 generates medical information (e.g., a blood flow change map) based on the difference data generated. The display controller 11 displays the generated medical information on the display device 2.

### Third Embodiment

The ophthalmic apparatus according to the present embodiment has a configuration for applying laser treatment to the fundus of the subject's eye. That is, the ophthalmic apparatus according to the present embodiment can be used as an ophthalmic laser treatment apparatus.

The ophthalmic apparatus according to the present embodiment may include the same configuration as conventional laser treatment apparatuses. Conventional laser treatment apparatuses are disclosed in, for example, Japanese Patent No. 5166454, Japanese Patent No. 5192383, Japanese Patent No. 5603774, Japanese Patent No. 5956883, and Japanese Patent No. 6067724. Any known techniques including the documents listed here may be applied to the present embodiment.

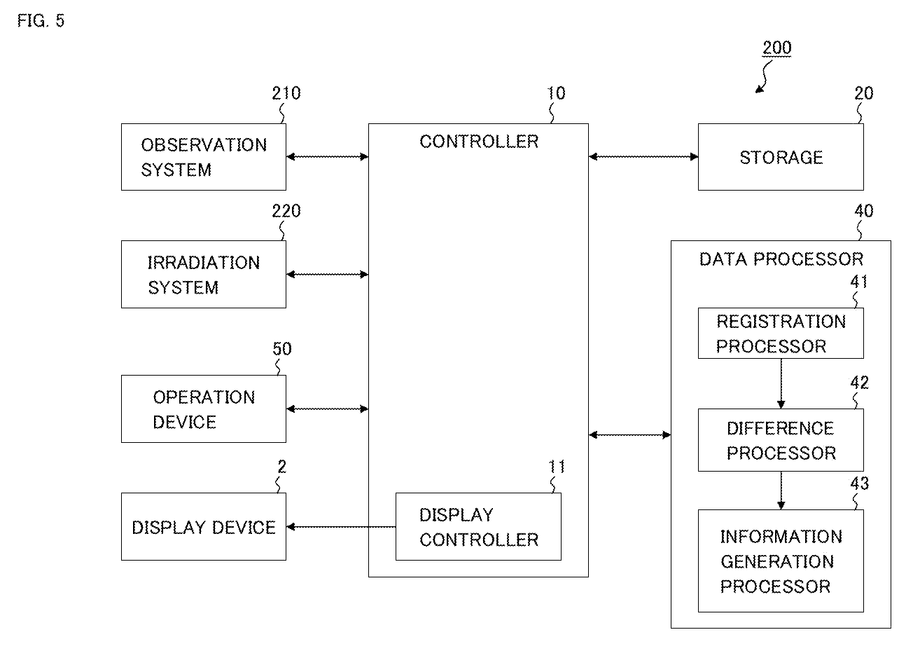

FIG. 5 shows an example of the configuration of the ophthalmic apparatus according to the present embodiment. Unlike the configuration of the first embodiment shown in FIG. 1, the ophthalmic apparatus 200 shown in FIG. 5 is provided with the observation system 210 and the irradiation system 220. The operation of the observation system 210 and that of the irradiation system 220 are controlled by the controller 10.

The observation system 210 provides an observation image of the fundus of the subject's eye to the user. The observation image is, for example, an image of the eye fundus provided to the user via an eyepiece such as a slit lamp microscope, or a moving image acquired by a fundus imaging apparatus such as a fundus camera or a scanning laser ophthalmoscope (SLO) and displayed in real time.

The observation system 210 may include the first optical system, the second optical system, and the third optical system. The first optical system projects illumination light onto the eye fundus. The second optical system guides, to the eyepiece, the returning light of the illumination light projected onto the eye fundus by the first optical system. The third optical system guides, to an imaging apparatus (a video camera), the returning light of the illumination light projected onto the eye fundus by the first optical system. The second optical system allows the user to observe the eye fundus through the eyepiece. In addition, the display controller 11 displays a moving image obtained by the video camera of the third optical system on the display device 2 as a movie.

The irradiation system 220 irradiates the fundus of the subject's eye with laser light. The irradiation system 220 includes a light source unit that generates a laser beam and an optical system that guides the laser beam generated by the light source unit to the eye fundus.

The light source unit of the irradiation system 220 includes an aiming light source that generates aiming light for aiming at a site to be subjected to laser treatment, and a treatment light source that emits treatment laser light. The operation of the light source unit is controlled by the controller 10. The aiming light and the treatment laser light are collectively referred to as irradiation light.

In the case where a configuration of performing aiming while observing the subject's eye via the eyepiece is applied, a light source (e.g., a laser light source, a light emitting diode) that emits visible light recognizable by the user is used as the aiming light source. In addition, in the case where a configuration of performing aiming while observing a displayed image of the subject's eye is applied, a light source (e.g., a laser light source, a light emitting diode) that emits light of a wavelength band detectable by an imaging apparatus for acquiring an image to be displayed is used as the aiming light source.

The treatment laser light may be a visible laser light or an invisible laser light depending on the application thereof. Further, the treatment light source may include a single laser light source that emits a plurality of pieces of laser light of mutually different wavelength bands, or a plurality of laser light sources.

An optical system of the irradiation system 220 guides, to the subject's eye, the irradiation light transmitted from the light source unit to the slit lamp microscope via an optical fiber, for example. The optical system includes an optical scanner. The optical scanner deflects the irradiation light in a two dimensional manner. The optical scanner includes, for example, a two dimensional optical scanner or two pieces of one dimensional optical scanners. The optical scanner includes any type of optical scanner such as a galvano scanner, or a MEMS optical scanner. The operation of the optical scanner is controlled by the controller 10.

The ophthalmic apparatus 200 can apply the irradiation light to the patient's eye E according to a pattern designated in advance. The projection image of the irradiation light is referred to as a spot. The irradiation light has various conditions (irradiation conditions). Exemplary irradiation conditions include the followings: the arrangement pattern of a plurality of spots (arrangement condition); the size of an arrangement pattern (arrangement size condition); the orientation of an arrangement pattern (arrangement orientation condition); the size of a spot (spot size condition); the interval between spots (spot interval condition); the intensity of irradiation light (power condition); the wavelength of irradiation light (wavelength condition); and the time length of the application of irradiation light (irradiation time condition). The controller 10 controls the operation of the irradiation system 220 according to the irradiation conditions. In addition, the display controller 11 can display information indicating the irradiation conditions on the display device 2.

Typically, in the preoperative planning of laser treatment for the subject's eye, treatment target position information representing treatment target positions (that is, positions in the eye fundus that are to be irradiation targets of treatment laser light) is generated. The treatment target position information is stored in the storage 20. The positions represented by the treatment target position information are defined as coordinates in the angiographic image data (the first angiographic image data) referred to in the preoperative planning, for example. The first angiographic image data is stored in the storage 20.

The registration processor 41 performs registration between the first angiographic image data and the second angiographic image data. The difference processor 42 generates difference data between the first angiographic image data and the second angiographic image data. The information generation processor 43 generates medical information based on the difference data generated.

Further, for example, the positions indicated in the treatment target position information can be represented using the coordinates in the difference data, by referring to the result of the registration between the first angiographic image data and the second angiographic image data. Furthermore, the positions indicated in the treatment target position information can be associated with positions in the medical information (map).

The display controller 11 can display, on the display device 2, an irradiation target position image indicating the irradiation target positions of treatment laser light and a difference image constructed from the difference data generated by the difference processor 42.

The difference image may be, for example, an image representing the difference data or a map image representing the medical information. The display controller 11 determines the display position of the irradiation target position image and the display position of the difference image based on the association relationship of positions between the treatment target position information and the difference data (or the medical information). In other words, a relative display position between the irradiation target position image and the difference image is determined.

The ophthalmic apparatus 200 is configured to provide the irradiation target position image and the difference image together with the observation image to the user.

In the case where the observation image is provided to the user via the eyepiece such as a slit lamp microscope, light output from the display device 2 enters the optical path of the second optical system that guides the returning light of the illumination light projected onto the fundus to the eyepiece, and is guided to the eyepiece. At this time, the optical path of the light output from the display device 2 is coupled to the optical path of the second optical system by a beam splitter (an optical path coupling member), for example. With such a configuration, the user can observe, via the eyepiece, the irradiation target position image and the difference image displayed on the display device 2 as well as the observation image of the eye fundus.

In the case where an observation image that is a real-time moving image acquired by a fundus imaging apparatus is provided to the user, the observation image, the irradiation target position image, and the difference image are displayed on the display device 2. In this case, the display device 2 is, for example, a display provided on a housing of the ophthalmic apparatus 200 or a peripheral device connected to the ophthalmic apparatus 200.

### Actions and Effects

Actions and effects of the ophthalmic apparatus according to the exemplary embodiment will be described.

An ophthalmic apparatus (1, 100, 200) according to the exemplary embodiment includes a storage (20) and a difference processor (42). The storage stores a plurality of pieces of angiographic image data acquired by applying optical coherence tomography (OCT) angiography to the fundus of a subject's eye a plurality of times. The difference processor generates difference data between the first angiographic image data and the second angiographic image data both read out from the storage.

Such an embodiment is capable of obtaining new information representing time-dependent changes such as the distribution of blood vessels, the state of blood vessels, and blood flow, from the difference data of different pieces of angiographic image data.

In the exemplary embodiment, the ophthalmic apparatus (1, 100, 200) may further include a registration processor (41) that performs registration between the first angiographic image data and the second angiographic image data. The difference processor (42) can generate the difference data from the first angiographic image data and the second angiographic image data to which the registration has been applied.

Such a configuration makes it possible to generate proper difference data even when the position matching between the first angiographic image data and the second angiographic image data has not been performed.

In the exemplary embodiment, the ophthalmic apparatus (1, 100, 200) may further include a first information generation processor (the information generation processor 43) that generates blood flow change information (e.g., a blood flow map) representing a change in blood flow in the fundus, based on the difference data generated by the difference processor (42).

With such a configuration, new information representing the time-dependent changes in the blood flow can be obtained.

In the exemplary embodiment, the ophthalmic apparatus (1, 100, 200) may further include a second information generation processor (the information generation processor 43) that generates blood vessel diameter change information (e.g., a blood vessel diameter change map) representing a change in a blood vessel diameter in the fundus, based on the difference data generated by the difference processor (42).

According to such a configuration, new information representing the time-dependent changes in the blood vessel diameter can be obtained.

In the exemplary embodiment, the ophthalmic apparatus (100) may further include a data acquisition device (110) that acquires data by applying OCT angiography to the fundus, and a data processor (120) that processes the acquired data to construct angiographic image data. The storage (20) can store the angiographic image data constructed by the data processor.

Such a configuration makes it possible to generate the difference data using the angiographic image data acquired by the ophthalmic apparatus, and to generate various kinds of medical information.

In the exemplary embodiment, the ophthalmic apparatus (200) may include an observation system (210), an irradiation system (220), and a display controller (11). The observation system provides an observation image of the fundus to a user. The irradiation system irradiates the fundus with treatment laser light. The display controller displays an irradiation target position image indicating an irradiation target position of the treatment laser light and a difference image constructed from the difference data generated by the difference processor on a display device (the display device 2). Further, the ophthalmic apparatus (200) may be configured to provide the irradiation target position image and the difference image together with the observation image of the fundus to the user.

According to such a configuration, the user can refer to the irradiation target position of the treatment laser light and the time-dependent changes in blood vessel distribution, blood vessel state, or blood flow, while observing the fundus in real time with the observation image, when performing laser treatment. As a result, the user can easily grasp the relationship between the irradiation target position of the treatment laser light and the position where the blood vessel distribution, blood vessel state, or blood flow has changed over time (and/or the position where the blood vessel distribution, blood vessel state, or blood flow has not changed over time). In addition, the user can utilize the grasped information for the laser treatment.

With the ophthalmic apparatus according to the exemplary embodiment as described above, an ophthalmic image processing method according to an exemplary embodiment including the following steps can be realized: storing a plurality of pieces of angiographic image data acquired by applying optical coherence tomography (OCT) angiography to the fundus of a subject's eye a plurality of times; and generating difference data between the first angiographic image data and the second angiographic image data from among the plurality of pieces of angiographic image data.

A program for causing a computer to execute the ophthalmic image processing method according to such an exemplary embodiment can be configured. Furthermore, a non-transitory computer readable recording medium storing such a program can be configured.

While certain embodiments have been described, these embodiments have been presented by way of example only, and are not intended to limit the scope of the inventions. Indeed, the novel embodiments described herein may be embodied in a variety of other forms; furthermore, various omissions, substitutions, additions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ophthalmic apparatus comprising:
a storage that stores a plurality of pieces of angiographic image data acquired by applying optical coherence tomography (OCT) angiography to a fundus of a subject's eye a plurality of times; and
a difference processor that generates difference data between a first angiographic image data and a second angiographic image data both read out from the storage.

2. The ophthalmic apparatus of claim 1, further comprising a registration processor that performs registration between the first angiographic image data and the second angiographic image data,
wherein the difference processor generates the difference data from the first angiographic image data and the second angiographic image data to which the registration has been applied.

3. The ophthalmic apparatus of claim 1, further comprising a first information generation processor that generates blood flow change information representing a change in blood flow in the fundus based on the difference data generated by the difference processor.

4. The ophthalmic apparatus of claim 1, further comprising a second information generation processor that generates blood vessel diameter change information representing a change in a blood vessel diameter in the fundus based on the difference data generated by the difference processor.

5. The ophthalmic apparatus of claim 1, further comprising:
a data acquisition device that acquires data by applying OCT angiography to the fundus; and
a data processor that processes the data acquired by the data acquisition device to construct angiographic image data,
wherein the storage stores the angiographic image data constructed by the data processor.

6. The ophthalmic apparatus of claim 1, further comprising:
an observation system that provides an observation image of the fundus to a user;
an irradiation system that irradiates the fundus with treatment laser light; and
a display controller that displays an irradiation target position image indicating an irradiation target position of the treatment laser light and a difference image constructed from the difference data generated by the difference processor on a display device,
wherein the irradiation target position image and the difference image are provided together with the observation image to the user.

7. A method of processing an ophthalmic image, the method comprising:
storing a plurality of pieces of angiographic image data acquired by applying optical coherence tomography (OCT) angiography to a fundus of a subject's eye a plurality of times; and
generating difference data between a first angiographic image data and a second angiographic image data from among the plurality of pieces of angiographic image data.

8. A non-transitory computer readable recording medium storing a program causing a computer to execute the ophthalmic image processing method of claim 7.
